(19) United States
(12) Patent Application Publication
(10) Pub. No.: US 2021/0128339 A1
(43) Pub. Date: May 6, 2021

(54) ADJUSTABLE BOOT BRACE

(71) Applicant: William J. Keegan, San Jose, CA (US)

(72) Inventor: William J. Keegan, San Jose, CA (US)

(21) Appl. No.: 16/674,719

(22) Filed: Nov. 5, 2019

(51) Int. Cl. A61F 5/01

(52) U.S. Cl. CPC A61F 5/0127; A61F 2005/0172

(57) ABSTRACT

An adjustable boot brace adapted to stretch and support an inflamed tissue and/or inflamed plantar fascia within a person's foot while healing. The adjustable boot brace includes an outer shell being formed from plastic, an inner padding formed from soft foam, a toe plate, and a hinge that pivotally connects the toe plate to the outer shell so a person can adjust the position and angle of their toes. Five hook and loop straps are used to position and hold the adjustable boot brace to a person's leg and foot, including two for a calf portion, two for a foot portion, and one for the toe plate. Furthermore, at least one adjustable hook and loop strap is used to hold the toe plate at a chosen angle with respect to the outer shell in order to stretch and support the inflamed tissue and/or inflamed plantar fascia within a person's foot while using the adjustable boot brace for healing.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of boot braces adapted to stretch and support an inflamed tissue and/or inflamed plantar fascia within a person's foot while healing.

### 2. Description of the Related Art

When a person injures tissues within their foot it may become necessary to stretch and support those tissues during the healing process. The preexisting methods and systems rely on boots that offer means for pulling the person's toes upwardly but does not offer a means for keeping the person's foot stationary while pulling the person's toes upwardly. Therefore, the tissues will not being maximally and/or properly stretched while healing and thereby may not heal properly if at all. Various attempts have been made to solve the above-mentioned problems such as those found in U.S. Pat. Nos. 4,577,769 to Ahmad et al; 6,602,216 to Nordt, III; 8,226,589 to Darby, II et al; and 2005/0043662 to Price et al. These prior art references are representative of prior adjustable boot devices. None of the above inventions and patents, taken either singly or in combination, is seen to describe the invention as claimed.

### BRIEF SUMMARY OF THE INVENTION

In view of the foregoing disadvantages inherent in the known art, the present invention provides an adjustable boot brace. The general purpose of the present invention, which will be described subsequently in greater detail, is to provide a boot brace adapted to stretch and support an inflamed tissue and/or inflamed plantar fascia within a person's foot while healing.

The instant adjustable boot brace is adapted to help heal inflamed tissues and/or an inflamed plantar fascia within a person's foot. The boot brace has a plastic outer shell with a soft foam padding inside. There are hook and loop straps that can be adjusted for the most comfortable fit and adjust the angle of their toes.

The main feature of the instant boot brace that is an improvement over the prior art boot braces is the way the instant toe plate can isolate the ball of your foot and your toes to be in the exact position that is most comfortable for the person as they work on getting their inflamed tissue and/or inflamed plantar fascia healed, which is done with a hinge, strap, or strap that stretches.

The instant boot brace incorporates two adjustable hook and loop straps, one on each side of the boot brace, from the front of the boot brace to the top of the boot brace. With these two adjustable straps the person will be able to adjust their boot at the hinge, strap, or strap that stretches into different positions. This will give the person the most comfort and the biggest benefit to stretching the inflamed tissue and can increase the angle of their toes as needed, or decrease the angle of their toes as needed.

The overall structure of the boot brace is solid with an outer shell being formed from plastic, an inner part formed from a soft foam padding, a toe plate, and a hinge, strap, or a strap that stretches that keeps the toe plate pivotally attached to the outer shell as the person adjusts the position of their toes. The outer shell is used to hold a person's calf in position and the bottom part of the outer shell is to keep the person's foot held down so only their toes will move backwards and upwards. The two adjustable side straps will keep their toes in a desired angle that is required. As for comfort and support, five hook and loop straps are incorporated. Two for the calf, two for the foot, and one for the toes.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention constructed and operative according to the teachings of the present invention.

FIG. 1 shows a perspective view illustrating the adjustable boot brace according to an embodiment of the present invention with insert outside of boot shell.

FIG. 2 shows a perspective view the adjustable boot brace in use according to the embodiment of FIG. 1 with insert inside of boot shell.

FIG. 3 shows a perspective bottom view illustrating a first embodiment of the hinge of the adjustable boot brace according to the embodiment of FIG. 1.

FIG. 4 shows a perspective bottom view illustrating a second embodiment of the hinge of the adjustable boot brace according to the embodiment of FIG. 1.

The various embodiments of the present invention will hereinafter be described in conjunction with the appended drawings, wherein like designations denote like elements.

### DECRIPTION OF THE PREFERRED EMBODIMENT

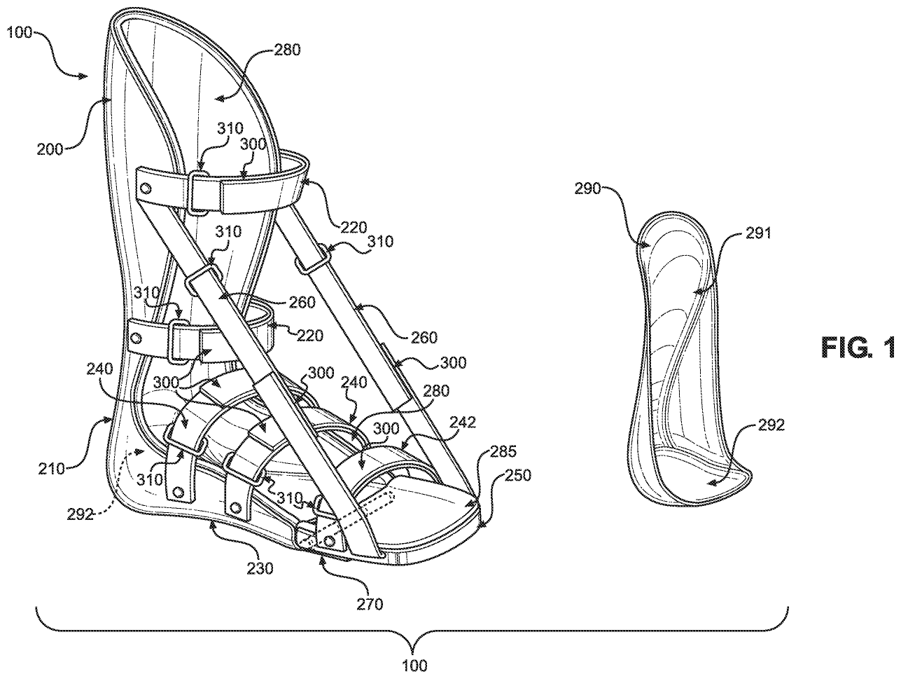

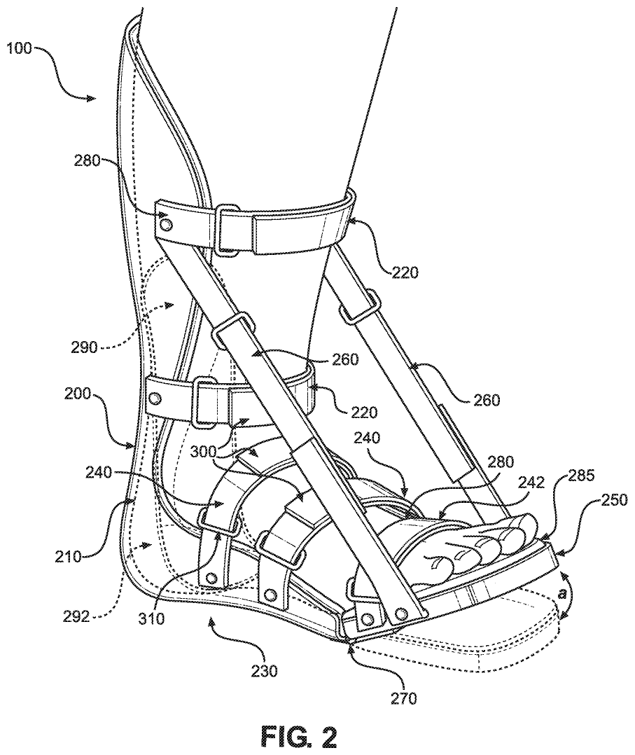

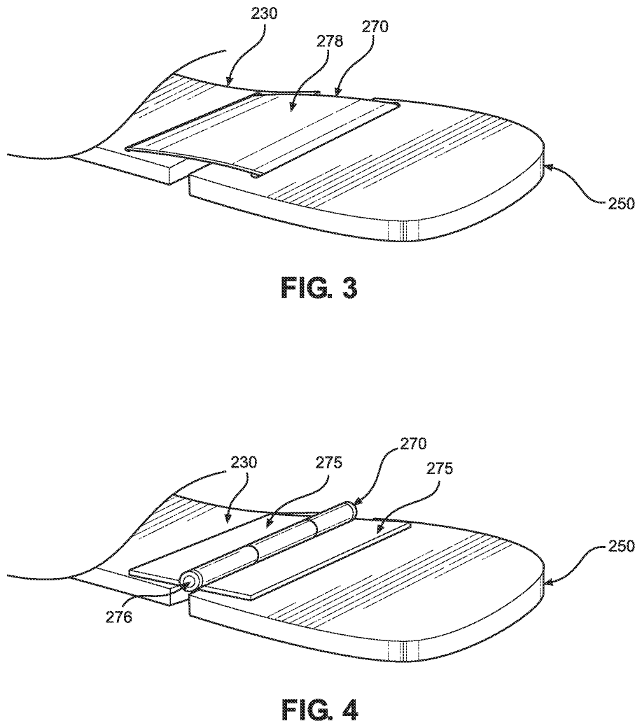

The preferred embodiment of the present invention relates to an adjustable boot brace adapted to help heal inflamed tissues and/or an inflamed plantar fascia within a person's foot. Referring now to FIGS. 1-4, an adjustable boot brace 100 is illustrated comprising an outer shell 200 including a rigid calf portion 210 that includes a proximal end, a distal end, a left side, a right side, and at least one adjustable strap 220, wherein the at least one adjustable strap is connected between the left side and the right side thereof, wherein the calf portion is adapted to be placed against and extend along a length of a user's calf, and the at least one adjustable strap is adapted to be placed against a front portion of the user's shin to thereby surround the user's leg and releasably hold the outer shell securely thereto; a rigid foot portion 230 including a proximal end, a distal end, a left side, a right side, and two adjustable straps 240, wherein the two adjustable straps are spaced from one another and connected between the left side and the right side thereof, wherein the foot portion is adapted to be placed against and extend along a length of the bottom of the user's foot, and the two adjustable straps are adapted to be placed against spaced top portions of the user's foot to thereby surround the user's foot and releasably hold the outer shell securely thereto, and wherein the proximal end of the foot portion is rigidly connected to the distal end of the calf portion forming an L-shape; a rigid toe plate 250 including a proximal end, a distal end, a left side, and a right side, wherein the toe plate is adapted to be placed against and extend along a length of the bottom of the toes of the user's foot, and wherein the proximal end of the toe plate is pivotally connected to the distal end of the foot portion 230, and at least one adjustable toe plate strap 260, wherein the at least one adjustable toe plate strap is attached between the rigid toe plate and the proximal end of the rigid calf portion, and is adapted to hold the rigid toe plate at a desired angle, as shown in FIG. 2 as angle “a”, with respect to the rigid foot portion. The adjustable boot brace 100 further comprises a hinge 270 connected between the rigid toe plate and the rigid foot portion, which may include two hinge plates 275 and a pivot pin 276 connected therebetween, or as a flexible, stretchable strap 278 connected therebetween, and is adapted to allow the toe plate to pivot with respect to the distal end of the foot portion.

In a preferred embodiment, there are two spaced adjustable straps 220 connected to the rigid calf portion 210; two adjustable toe plate straps 260, wherein a first adjustable toe plate strap 260 is connected between the left side of the proximal end of the rigid calf portion 210 and the left side of said distal end of said rigid toe plate 250, and wherein a second adjustable toe plate strap 260 is connected between the right side of said proximal end of said rigid calf portion 210 and the right said of said distal end of said rigid toe plate 250. The adjustable straps 220 of the rigid calf portion 210, the adjustable straps 240 of the rigid foot portion 230, and the adjustable straps 260 of the rigid toe plate 250 may be formed from a flexible material and include hook and loop fasteners 300 and length adjusting rings 310 adapted to adjust the lengths thereof respectively. Furthermore, the rigid toe plate 250 further includes an adjustable toe strap 242 connected between said left side and said right side thereof and adapted to hold the user's toes in position upon an upper surface thereof. And, wherein the adjustable toe strap 242 includes hook and loop fasteners 300 and a length adjusting ring 310 adapted to adjust the length thereof. The greatest advantage of the instant configuration over the prior art configurations is that the angle between the rigid toe plate 250 and the rigid foot portion 230, as shown in FIG. 2 as angle “a”, is adjustable and can be held in place by the at least one adjustable toe plate strap 260. The closest prior art configurations do not use “rigid” toe plates and simply pull on the tips of a user's toes thereby stretching the muscles, tendons, and ligaments of the toes instead of just the intended area of the plantar fascia area of the user's foot. Therefore, the instant invention is more efficient and will avoid stretching muscles, tendons, and ligaments within a user's toes when in use, especially if/when the user may also have injuries to their toes.

The outer shell and the toe plate may be formed from plastic, fiberglass, aluminum, or copper. The flexible straps may be formed from nylon or polyester. Furthermore, the rigid toe plate may be formed having a substantially rectangular shape.

The adjustable boot brace may further comprise an inner cushioning liner 280 connected to an inner surface of the outer shell and adapted to contact the user's calf and foot when in use, and a toe plate cushioning liner 285 connected to a top surface of said rigid toe plate and adapted to contact the user's toes when in use.

The adjustable boot brace 100 may further comprise at least one adjustment insert 290 adapted to be placed against an inner surface of the rigid calf portion 210 of the outer shell and adapted to adjust the positioning of the user's foot with respect to the rigid calf portion, the rigid foot portion, and the rigid toe plate. The adjustment insert 290 may include at least one curved portion 292 adapted to conform to the shape of the user's heel, and wherein the adjustment insert 290 may be formed from a semi-rigid material and include an outer cushioning layer 291 attached thereto. The at least one adjustment insert 290 may also be formed having other shapes and sizes to thereby adapt to the size and shape of the user's leg and foot. In other situations, a plurality of adjustment insert 290 may be used in conjunction with one another to fit the desired needs of the user.

It should be noted that the steps described in the method of use can be carried out in many different orders according to user preference. The use of “step of” should not be interpreted as “step for”, in the claims herein and is not intended to invoke the provisions of 35 U.S.C. § 112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. An adjustable boot brace comprising:
an outer shell including:
a rigid calf portion including:
a proximal end;
a distal end:
a left side;
a right side; and
at least one adjustable strap;
wherein said at least one adjustable strap is connected
between said left side and said right side thereof;
wherein said calf portion is adapted to be placed against and extend along a length of a user's calf, and said at least one adjustable strap is adapted to be placed against a front portion of the user's shin to thereby surround the user's leg and releasably hold said outer shell securely thereto;
a rigid foot portion including:
a proximal end;
a distal end:
a left side;
a right side; and
at least one adjustable strap;
wherein said at least one adjustable strap is connected between said left side and said right side thereof;
wherein said foot portion is adapted to be placed against and extend along a length of the bottom of the user's foot, and said at least one adjustable strap is adapted to be placed against a top portion of the user's foot to thereby surround the user's foot and releasably hold said outer shell securely thereto; and
wherein said proximal end of said foot portion is rigidly connected to said distal end of said calf portion forming an L-shape;
a rigid toe plate including:
a proximal end;
a distal end:
a left side; and
a right side;
wherein said toe plate is adapted to be placed against and extend along a length of the bottom of the toes of the user's foot; and
wherein said proximal end of said toe plate is pivotally connected to said distal end of said foot portion; and
at least on adjustable toe plate strap;
wherein said at least on adjustable toe plate strap is attached between said rigid toe plate and said proximal end of said rigid calf portion, and is adapted to hold said rigid toe plate at a desired angle with respect to said rigid foot portion.

2. The adjustable boot brace of claim 1, wherein said rigid outer shell is formed from a material chosen from a group of materials consisting of plastic, fiberglass, aluminum, and copper.

3. The adjustable boot brace of claim 2, wherein said rigid toe plate is formed from a material chosen from a group of materials consisting of plastic, fiberglass, aluminum, and copper.

4. The adjustable boot brace of claim 1, wherein there are two spaced adjustable straps connected to said rigid calf portion.

5. The adjustable boot brace of claim 1, wherein said at least one adjustable toe plate strap is attached between the proximal end of said rigid calf portion and said distal end of said rigid toe plate.

6. The adjustable boot brace of claim 1, wherein there are two adjustable toe plate straps, wherein a first adjustable toe plate strap is connected between the left side of said proximal end of said rigid calf portion and the left side of said distal end of said rigid toe plate; and wherein a second adjustable toe plate strap is connected between the right side of said proximal end of said rigid calf portion and the right said of said distal end of said rigid toe plate.

7. The adjustable boot brace of claim 1, further comprising a hinge connected between said rigid toe plate and said rigid foot portion.

8. The adjustable boot brace of claim 7, wherein said hinge includes two hinge plates and a pivot pin connected therebetween.

9. The adjustable boot brace of claim 7, wherein said hinge is formed as a flexible strap connected therebetween.

10. The adjustable boot brace of claim 9, wherein said flexible strap is formed from a material chosen from a group of materials consisting of nylon and polyester.

11. The adjustable boot brace of claim 1, wherein said at least one adjustable strap of said rigid calf portion, said at least one adjustable strap of said rigid foot portion, and said at least one adjustable strap of said rigid toe plate are formed from a flexible material and include hook and loop fasteners adapted to adjust the lengths thereof respectively.

12. The adjustable boot brace of claim 1, wherein said rigid toe plate is formed having a substantially rectangular shape.

13. The adjustable boot brace of claim 1, further comprising an inner cushioning liner connected to an inner surface of said outer shell and adapted to contact the user's calf and foot when in use.

14. The adjustable boot brace of claim 1, further comprising a toe plate cushioning liner connected to a top surface of said rigid toe plate and adapted to contact the user's toes when in use.

15. The adjustable boot brace of claim 1, further comprising at least one adjustment insert adapted to be placed against an inner surface of said rigid calf portion of said outer shell and adapted to adjust the positioning of the user's foot with respect to said rigid calf portion, said rigid foot portion, and said rigid toe plate.

16. The adjustable boot brace of claim 15, wherein said at least one adjustment insert includes at least one curved portion adapted to conform to the shape of the user's heel.

17. The adjustable boot brace of claim 15, wherein said at least one adjustment insert is formed from a semi-rigid material, and includes an outer cushioning layer attached thereto.

18. The adjustable boot brace of claim 1, wherein said rigid toe plate further includes an adjustable toe strap connected between said left side and said right side thereof and adapted to hold the user's toes in position upon an upper surface thereof.

19. The adjustable boot brace of claim 18, wherein said adjustable toe strap of said rigid toe plate is formed from a flexible material and includes hook and loop fasteners adapted to adjust the length thereof.

20. The adjustable boot brace of claim 1, wherein there are two spaced adjustable straps connected to said rigid foot portion.
